(19) United States
(12) Patent Application Publication
(10) Pub. No.: US 2009/0105202 A1
(43) Pub. Date: Apr. 23, 2009

(54) Androsterone Derivatives and Method of Use thereof

(75) Inventor: Li-Xi Yang, San Francisco, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignees: CALIFORNIA PACIFIC MEDICAL CENTER, San Francisco, CA (US); CATHOLIC HEALTHCARE WEST, San Francisco, CA (US)

(21) Appl. No.: 12/083,851

(22) PCT Filed: Oct. 18, 2006
(86) PCT No.: PCT/US2006/060051

§ 371 Date: Apr. 18, 2008

Related U.S. Application Data
(60) Provisional application No. 60/729,463, filed on Oct. 20, 2005.

(51) Int. Cl. A61K 31/58; C07J 1/00; A61K 31/5685; A61P 35/00; C07J 43/00

(52) U.S. Cl. 514/176; 552/641; 514/178; 540/108; 540/113

(57) ABSTRACT

The disclosure provides androsterone derivatives. The derivatives of the disclosure are useful in the treatment of androgen- and estrogen-associated diseases and disorders, including breast cancer.

### CROSS REFERENCE TO RELATED APPLICATIONS

This application claims priority to Provisional Application Ser. No. 60/729,463, filed Oct. 20, 2005, the disclosure of which is incorporated herein by reference.

### STATEMENT REGARDING FEDERALLY SPONSORED RESEARCH

The present invention was funded in part by Grant No. DAMD17-99-1-9018 awarded by DOD. The government may have certain rights in the invention.

### FIELD OF THE INVENTION

The disclosure relates to novel steroids and more particularly to androsterone derivatives useful as anti-cancer, anti-obesity, anti-diabetic and hypolipidemic agents.

### BACKGROUND

Dehydroepiandorsterone (DHBA) and dehydroepiandrosierone-sulfate are major adrenal secretory products in many mammalian species. Although DHEA-sulfate is the main precursor of placental estrogen and is converted into active androgens in peripheral tissue, there is no strong biological role for either DHBA or DHEA-sulfate in the normal subject. Several studies suggest that these steroids are associated with cell proliferative disorders as well as other androgen-associated diseases and disorders.

Examples of androgen-associated diseases and disorders include, but are not limited to, prostate cancer, benign prostatic hyperplasia, acne, seborrhea;, hirsutism, androgenic alopecia, precocious puberty., adrenal hyperplasia, and polycystic ovarian syndrome. In addition, estrogen-associated diseases and disorders can be included such as, for example, breast cancer, endometriosis, leiomyoma, and precocious puberty.

### SUMMARY

The disclosure provides androsterone derivatives, methods of synthesis and methods of use in the treatment of various androgen- and estrogen-associated diseases and disorders.

In one aspect the androsterone derivatives inhibit breast cancer cell growth via counteracting the effect of female hormones and/or binding on receptors for such hormones (e.g. as antagonists) inducing inhibition of cellular proliferation and inducing killing of ceils having cell proliferative disorders associated with androgens and estrogens.

In another aspect the disclosure provides androsterone ester compounds, pharmaceutical compositions of these androsterone derivatives, methods of using androsterone derivatives (e.g., for the treatment of cancer).

In yet another aspect, the disclosure provides androsterone-camptothecin combination compounds, pharmaceutical compositions of these androsterone derivatives., methods of using androsterone derivatives (e.g., for the treatment of cancer).

The details of one or more embodiments are set forth in the accompanying drawings and the description below. Other features, objects, and advantages will he apparent from the description and drawings, and from the claims.

### BRIEF DESCRIPTION OF THE FIGURE

FIG. 1 shows the effect of an androsterone derivative of the disclosure on cancer cells.

### DETAILED DESCRIPTION

As used herein and in the appended claims, the singular forms “a” “and,” and “the” include plural referents unless the contest clearly dictates otherwise. Thus, for example, reference to “an antigen” includes a plurality of such antigens and reference to “the immune cell” includes reference to one or .more immune cells known to those skilled in the art, and so forth.

Unless defined otherwise, ah technical and scientific terms used herein have the same meaning as commonly understood to one of ordinary skill, in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Androsterone is a steroid hormone excreted in urine that reinforces masculine characteristics having the general formula as set forth in Formula I:

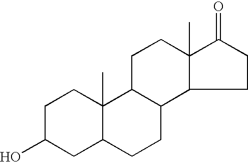

The disclosure provides androsterone derivatives having the general formulas II and III:

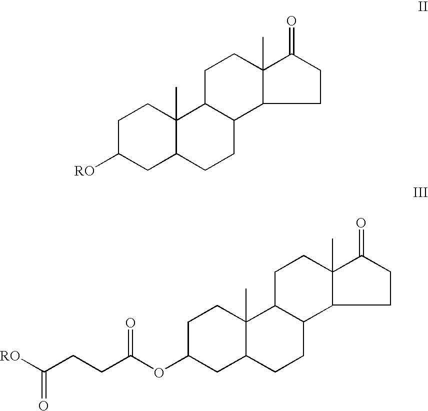

In one aspect the R groups are aromatic (e.g., aromatic nitrogen-containing heterocycles). In some aspect, the R groups have extended aromatic systems with electron withdrawing groups (e.g., electrophilic). Aromatic nitrogen-containing heterocycles, typically contain a 5- or 6-membered monocyclic substituent, or a bicyclic fused or linked 5- or 6-membered ring, such as imidazolyl, indolyl, pyridinyl, pyrimidinyl, pyrrolyl, quinolinyl, tetrazolyl, 1,2,4-triazolyl, and the like.

Aromatic nitrogen-containing heterocycles include, by way of example, 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, 2,4,6-trimethylpyridine, and combinations thereof.

In another aspect, the R group is a non-aromatic nitrogen-containing heterocyle. Non-aromatic nitrogen-containing heterocycles, typically contain 4- to 6-membered rings such as acetimido, morpholinyl, lactams and imides (e.g., γ-butyrolactam, ε-caprolactam, N-phenyl-β-propiolactam), phthalimido, piperidyl, piperidino, piperazinyl, pyrrolidinyl, succinimido, aid the like. Non-aromatic nitrogen-containing heterocycles include, by way of example, 1,2-dimethylpiperidine, 2,5-dimethylpiperazine, 1,2-dimethylpyrrolidine, 1-ethylpiperidine, n-methylpyrrolidine, morpholine, piperazine, piperidine, pyrrolidine, 2,2,6,6-tetramethylpiper-idine, 2,2,4-trimethylpiperidine, and combinations thereof. In some more particular aspect, the R group is an atropine or a scopolamine.

In another aspect, the methods of the disclosure utilize heterocyclic compounds in the synthesis of the androsterone derivatives of the disclosure.

In yet another aspect, the methods of the disclosure utilize camptothecin compounds in the synthesis of the androsterone derivatives of the disclosure.

In one aspect of the disclosure, the androsterone derivative comprises a compound having the general formula II:

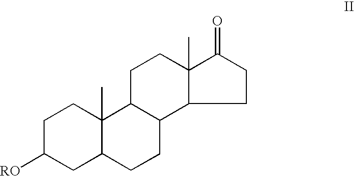

wherein R is selected from the group consisting of:

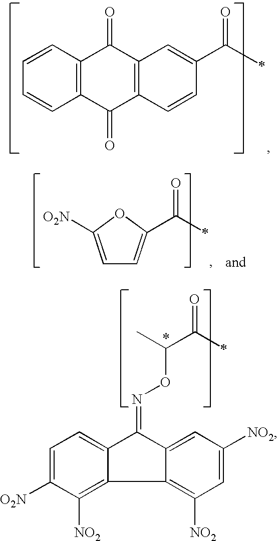

wherein * indicates the (+) chiral center in the original molecule.

In another aspect of the disclosure, the androsterone derivative comprises a compound having the general formula III:

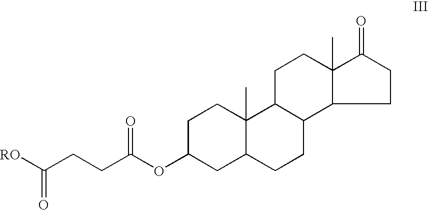

wherein R is selected from the group consisting of:

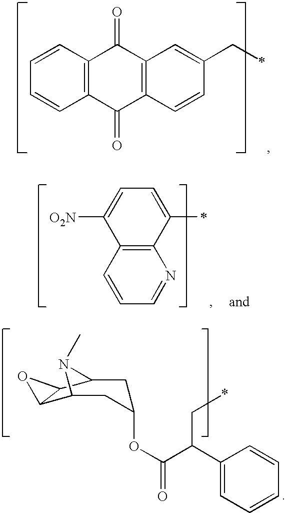

One aspect of the disclosure is a pharmaceutical composition useful for treating an androgen-associated disease in a warm-blooded animal which composition comprises compound of the disclosure as defined herein in combination with a pharmaceutically acceptable excipient. The composition is prepared in accordance with known formulation techniques to provide a composition suitable for oral, topical, transdermal, rectal, by inhalation, parenteral (intravenous, intramuscular, or intraperitoneal) administration, and the like. Detailed guidance for preparing compositions of the disclosure are found by reference to the 18.sup.th or 19.sup.th Edition of Remington's Pharmaceutical Sciences, Published by the Mack Publishing Co., Easton, Pa. 18040.

Unit doses or multiple dose forms are contemplated, each offering advantages in certain clinical settings. The unit dose would contain a predetermined quantity of active compound calculated to produce the desired effect(s) in the setting of treating disease. The multiple dose form may be particularly useful when multiples of single doses, or fractional doses, are required to achieve the desired ends. Either of these dosing forms may have specifications that are dictated by or directly dependent upon the unique characteristic, of the particular compound, the particular therapeutic effect to be achieved, and any limitations inherent in the art of preparing the particular compound for treatment of cancer.

The compound may he administered orally in a suitable formulation as an ingestible tablet; a buccal tablet, capsule, caplet elixir, suspension, syrup, trouche, wafer, lozenge, and the like. Generally, the most straightforward, formulation is a tablet or capsule (individually or collectively designated as an “oral dosage unit”). Suitable formulations are prepared in accordance with a standard formulating techniques available that match the characteristics of the compound to the excipients available for formulating an appropriate composition.

The form may deliver a compound rapidly or may be a sustained-release preparation. The compound may be enclosed in a hard or soft capsule, may be compressed into tablets, or may be incorporated with beverages, food or otherwise into the diet.

The suitable formulation of an oral dosage unit may also contain: a binder, such as gum tragacanth, acacia, corn starch, gelatin; sweetening agents such as lactose or sucrose; disintegrating agents such as com starch, alginic acid and the like; a lubricant such as magnesium stearate; or flavoring such a peppermint, oil of wintergreen or the like. Various other material may be present as coating or to otherwise modify the physical, form of the oral dosage unit. The oral dosage unit may be coated with shellac, a sugar or both. Syrup or elixir may contain the compound, sucrose as a sweetening agent, methyl and propylparabens as a preservative, a dye and flavoring. Any material, utilized should be pharmaceutically-acceptable and substantially non-toxic. Details of the types of excipients useful may be found in the nineteenth edition of “Remington: The Science and Practice of Pharmacy,” Mack Printing Company, Easton, Pa. See particularly chapters 91-93 for a fuller discussion.

A compound may be administered parenterally, e.g., intravenously, intramuscularly, intravenously, subcutaneously, or interperitonically. The carrier or excipient or excipient mixture can be a solvent or a dispersive medium containing, for example, various polar or non-polar solvents, suitable mixtures thereof, or oils. As used herein “carrier” or “excipient” means a pharmaceutically acceptable carrier or excipient and includes any and all solvents, dispersive agents or media, coating(s), antimicrobial agents, iso/hypo/hypertonic agents, absorption-modifying agents, and the like. The use of such substances and the agents for pharmaceutically active substances is well known in the art. Except insofar as any conventional media or agent is incompatible with the active ingredient, use in therapeutic compositions is contemplated. Moreover, other or supplementary active ingredients can also be incorporated into the final composition.

Solutions of the compound may be prepared in suitable diluents such as water, ethanol, glycerol, liquid polyethylene glycol(s), various oils, and/or mixtures thereof, aid others known to those skilled in the art.

The pharmaceutical forms suitable for injectable use include sterile solutions, dispersions, emulsions, and sterile powders. The final form must, be stable under conditions of manufacture and storage. Furthermore, the final pharmaceutical form must he protected against contamination and must therefore, be able to inhibit the growth of microorganisms such as bacteria or fungi. A single intravenous or intraperitoneal dose can be administered. Alternatively, a slow long term infusion or multiple short term daily infusions may be utilized, typically lasting from 1 to 8 days. Alternate day or dosing once every several days may also be utilized.

Sterile, injectable solutions are prepared by incorporating a compound in the required amount into one or more appropriate solvents to which other ingredients, listed above or known to those skilled in the art, may be added as required. Sterile injectable solutions are prepared by incorporating the compound in the required amount in the appropriate solvent with various other ingredients as required. Sterilizing procedures, such as filtration, then follow. Typically, dispersions are made by incorporating the compound into a sterile vehicle which also contains the dispersion medium and the required other ingredients as indicated above. In the case of a sterile powder, die preferred methods include vacuum drying or freeze drying to which any required ingredients are added.

In all cases the final form, as noted, must be sterile and must also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

Prevention or inhibition of growth of microorganisms may be achieved through the addition of one or more antimicrobial agents such as chlorobutanol, ascorbic acid, parabens, thermerosal, or the like. It may also he preferable to include agents that alter the tonicity such as sugars or salts.

In some cases, e.g., where a compound of the disclosure is quite water insoluble, it may be useful to provide liposomal delivery. The system restrains the compound of the disclosure by incorporating, encapsulating, surrounding, or entrapping the compound of the disclosure in, on, or by lipid vesicles or liposomes, or by micelles.

In addition, the disclosure provides methods of using the androsterone derivatives in the treatment of androgen-associate diseases and disorders including estrogen-associated diseases and disorders.

An androgen-associated disease or disorder includes, for example, prostate cancer, benign prostatic hyperplasia, acne, seborrhea, hirsutism, androgenic alopecia, precocious puberty, adrenal hyperplasia, and polycystic ovarian syndrome. In addition, estrogen-associated diseases and disorders can be included such as, for example, breast cancer, endometriosis, leiomyoma, and precocious puberty.

Precocious puberty is usually associated with an excess of androgen secretion, usually of adrenal origin. Current treatments include a blockade of adrenal secretion by glucocorticoids. Another treatment is the use of LHRH agonists to cause medical castration.

Polycystic ovarian syndrome is associated with an excess of androgen secretion by the ovaries. LHRH agonists are used among other, as treatment to cause medical castration.

Androgenic and estrogenic activity may be suppressed by administering androgen receptor antagonists (“antiandrogens”) or estrogen receptor antagonists (“antiestrogens”), respectively. See e.g. WO 94/26767 and WO 96/26201. Androgenic and estrogenic activity may also be reduced by inhibiting receptor activation using receptor antagonists, suppressing androgen or estrogen biosynthesis using inhibitors of enzymes that catalyze one or more steps of such biosynthesis or by suppressing ovarian or testicular secretions by known methods.

Both androgen-related and estrogen-related diseases and disorder may be treated with an androsterone derivative of the disclosure. Androgen-sensitive diseases are those whose onset or progress is aided by androgen activation of androgen receptors, and should respond favorably to treatment with an androsterone derivative of the disclosure because of the reduction of androgen biosynthesis that is achieved thereby. Estrogen-sensitive diseases (diseases whose onset or progress is aided by activation of the estrogen receptor) should also benefit because many androgens whose biosynthesis is suppressed by the compound(s) of the disclosure are precursors to estrogens, and the compound(s) may therefore reduce estrogen biosynthesis as well Androgen-sensitive diseases include but are not limited to prostatic cancer, benign prostatic hyperplasia, acne, seborrhea, hirsutism, androgenic alopecia, and polycystic ovarian syndrome. Estrogen-sensitive diseases include but are not limited to breast cancer, endometrial cancer, endometriosis, and endometrial leiomyoma.

In some aspects of the disclosure it may be desirable to inhibit estrogen activity while maintaining androgen activity. For example, breast cancer (and some other estrogen-sensitive diseases, e.g. ovarian cancer, uterine cancer, and endometrial cancer) respond favorably to androgens. Therefore, a compound which inhibits estrogen activity, and which is also androgenic, car be especially useful for the treatment of breast cancer and other diseases which respond negatively to estrogen and positively to androgen.

In some aspects of the disclosure it may be desirable to promote cancer cell death using androgen-camptothecin combination compounds wherein the camptothecin component exhibit additive anticancer activity. It has been shown that camptothecin inhibits topoisomerase, an enzyme that, is required for its sniveling and relaxation of DNA during molecular events such as replication and transcription.

The androsterone derivatives in accordance with the disclosure can be utilized as part of a combination therapy with other strategies, which modulate androgen- or estrogen-associated diseases and disorders through other mechanisms, thus providing synergistic combinations. For example, a combination therapy can include an androsterone derivative of the disclosure in combination with an agent selected from the group consisting of LHRH agonists (see, e.g., U.S. Pat. Nos. 4,659,695 and 4,666,883); FLUTAMIDE (N-[4-nitro-3-(trifluoromethyl)phenyl)]-2-methyl propanamide), NILUTAMIDE, or CASODEX; antiestrogens (e.g., EM-800 reported in PCT/CA96/00097; TAMOXIFEN ((Z)-2-[4-(1,2-diphenyl-1-butenyl)]-N,N-dimethylethanamine) and ICI 182780 (available from Zeneca, UK), TOREMIFENE (available from Orion-Farmos Pharmaceutical, Finland), DROLOXIFENE (Pfizer Inc., USA), RALOXIFENE (Eli Lilly aid. Co., USA), LY 335563 and LY 353381 (Eli Lilly and Co., USA), LODOXIFENE (SmithKline Beecham, USA), LEVORMELOXIFENE (Novo Nordisk, A/S, Denmark): TRILOSTANE (2α-cyano-4α,5α-epoxy-17β-hydroxyandrostan-3-one); inhibitors of testosterone 5-alpha-reductase (e.g., PROSCAR); an aromatase inhibitor (e.g., ARIMIDEX); and androgenic compounds (e.g., medroxyprogesterone acetate, and megestrol acetate).

In general, for both androgen-associated diseases and estrogen-associated diseases, simultaneous treatment with inhibitors of sex steroid biosynthesis (inhibitors of enzymes which catalyze one or more steps of estrogen, or androgen biosynthesis), and with estrogen receptor antagonists and/or androgen receptor antagonists, are believed to have additive rather than redundant effect because they are acting in a beneficial manner by a different mechanism.

Different sex steroid-dependent diseases respond differently to both androgen receptor activation and estrogen receptor activation. For example, breast career responds unfavorably to estrogen receptor activation, but favorably to androgen receptor activation. On the other hand, benign prostatic hyperplasia responds unfavorably to activation of either the estrogen or androgen receptor,

When an androsterone derivative of the disclosure is used, either alone or as part of one of tire combination therapies described herein, the attending clinician will typically target the subject's serum concentration between 0.5 ng/ml and 100 ng/ml, more typically between 1 ng/ml and 20 ng/ml, and more commonly between 1 ng/ml and 10 ng/ml. Serum concentration may be measured by various techniques known in the art (e.g., LC/MS). When administered orally, the dosage which is usually effective to provide the desired serum levels is between 1.0 mg and 1,000 mg of active ingredient per day per 50 kg of body weight, typically between 10 mg and 500 mg and more commonly between 10 mg and 100 mg. However, dosage will vary with the bioavailability of the chosen inhibitor and with individual subject's response. The attending clinician will typically monitor an individual subject's response and metabolism and adjust tire subject's dosage accordingly. When administered by injection, a lower dosage is typically used, e.g. 10 mg to 100 mg per day per 50 kg of body weight.

All of the active ingredients (including the androsterone derivatives of the disclosure) used in any of the therapies discussed herein may be formulated in pharmaceutical compositions which may include one or more additional active ingredients as discussed above. Alternatively, they may each be administered individually separately or simultaneously. In some embodiments of the disclosure, one or more active ingredients are formulated in a single pharmaceutical composition.

The working examples below are provided to illustrate, not limit the disclosure. Various parameters of the scientific methods employed in, these examples are described in detail below and provide guidance for practicing the disclosure in

### EXAMPLES

The following examples are given to provide representative compounds included as part of this disclosure. The examples also provide descriptions of in vitro and in vivo assays to aid in determining the utility of the compounds. Throughout the examples chemical formulas will be used to name compounds as appropriate.

### 1. (3β, 5α)-3-Hydroxyandrostan-17-one 9,10-dihydro-9,10-dioxo-2-anthracenecarboxylate (990624)

The reaction mixture of epiandrosierone (130 mg, 0.45 mmol), anthraquinone-2-carbonyl chloride (135 mg, 0.5 mmol), triethylamine (100 mg, 1.0 mmol) and dichloromethane (6.0 ml) were stirred at room temperature for 20 h. Then 20 ml of dichloromethane was added. Organic layer was washed with water (20 ml), saturated NaHCO₃ solution (15 ml) and brine (20 ml), and then dried over MgSO₄. After the solvent was removed in vacuo, the resulting solid was recrystallized from ethyl alcohol and ethyl acetate to give 46 mg (3β, 5α)-3-Hydroxyandrostan-17-one 9,10-dihydro-9,10-dioxo-2-anthracenecarboxylate.

The chemical structure analysis was performed by ¹HNMR (CDCl₃, 600 MHz); δ 8.93 (s, 1H, Ar—H), 8.39-8.43 (m, 4H, Ar—H), 7.84 (s, 2H, Ar—H), 5.05 (s, 1H, H-3), 2.45 (t, 1H-16), 2.20-0.70 (m, 21H, ), 0.94 (s, 3H, CH₃-19), 0.88 (s, 3H, CH₃).

### 2. (3β,5α)-3-Hydroxyandrostan-17-one 5-nitro-2-furoate (991027)

The reaction mixture of 5-nitro-2-furoic acid (157 mg, 1.0 mmol), epiandrosterone (163 mg, 0.60 mmol), EDCI (200 mg, 1.05 mmol), DMAP (20 mg, 0.2 mmol) and THF (4 ml) was stirred at room temperature for 6.5 h. Evaporation of THF gave a residue which was dissolved in dichloromethane (20 ml). The organic layer of dichloromethane was washed with H₂O, 5% Na₂CO₃, H₂O and brine, and then dried over MgSO₄. The solvent was removed under vacuum. The resulting solid was recrystallized from C₂H₅OH to give 180 mg (73.2%) (3β,5α)-3-hydroxyandrostan-17-one 5-nitro-2-furoate 134 mg.

The chemical structure analysis was performed by ¹HNMR (CDCl₃, 600 MHz): δ 7.35 (s, 1H, Ar—H), 7.28(s, 1H, Ar—H), 4.99 (s, 1H, H-3), 2.45 (t, 1H, H-16), 2.20-0.70 (m, 21H, ), 0.90 (s, 3H, CH₃-19), 0.87 (s, 3H, CH₃).

### 3. (3β,5α)-3-Hydroxyandrostan-17-one (+)-2-(2,4,5,6-tetranitro-9-flourenylidene-aminooxy)proprionate (991022)

The reaction mixture of (+)-2-(2,4,5,6-tetranitro-9-flourenylidene-aminooxy)-proprionic acid (224 mg, 0.5 mmol), epiandrosterone (122 mg, 0.45 mmol), DCC (123 mg, 0.6 mmol), DMAP (10 mg, 0.1 mmol) and THF (6 ml) was stirred at room temperature for 6 h. Then 4 drops of water were added to above solution. After stirred for 10 min, the mixture was filtered and the solid was washed with THF. Evaporation of THF gave a residue which was dissolved in dichloromethane (20 ml). The organic layer of dichloromethane was washed, with H₂O, 5% Na₂CO₃, H₂O and brine, and then dried over MgSO₄. The solvent was removed under vacuum. The resulting solid was recrystallized from ethyl acetate and petroleum ether to give 1.49 mg (47.3%) (3β,5α)-3-hydroxyandrostan-17-on(+)-2-(2,4,5,6-tetranitro-9-flourenylidene-aminooxy)-proprionate.

The chemical structure analysis was performed by ¹HNMR (CDCl₃, 600 MHz): δ 9.59 (s, 1H, Ar—H), 8.97 (d, 2H, Ar—H), 8.90 (s, 1H, Ar—H), 5.22 (q, 1H, CHCO), 4.88 (m, 1H, CHO), 2.45 (t, 1H, H-16), 2.20-0.70 (m, 21H, ), 1.83 (d, 3H, CH3), 0.88 (s, 3H, CH₃-19), 0.86 (s, 3H, CH₃).

### 4. 1-((3β,5α)-3-Hydroxyandrostan-17-one)-4-(2-(hydroxymethyl)anthraquinone)-succinate (991120)

The reaction mixture of (3β,5α)-3-Hydroxyandrostan-17-one monosuccinate (25 mg, 0.064 mmol), 2-(hydroxymethyl) anthraquinone (16 mg, 0.067 mmol), EDCI (20 mg, 0.11 mmol), DMAP (2 mg, 0.02 mmol) and dichloromethane (4 ml) was stirred at room temperature for 6 h. Then 20 ml of dichloromethane was added. Organic layer was washed, with H₂O, 5% Na₂CO₃, H₂O and brine, and then dried over MgSO₄. The solvent was removed under vacuum to give 37 mg 1-((3β,5α)-3-Hydroxyandrostan-17-one)-4-(2-(hydroxymethyl) anthraquinone)succinate as yellow solid.

The chemical structure analysis was performed by ¹HNMR (CDCl₃, 600 MHz): δ 8.30(m, 4H, Ar—H), 7.80 (m, 3H, Ar—H), 5.30 (s, 2H, ArCH₂O), 4.69 (m, 1H, CHO), 2.74 (t, 2H, CH₂), 2.64 (t, 2H, CH₂), 2.41 (m, 1H, H₁₆), 2.20-0.67 (m, 21H,), 1.83 (d, 3H, CH₃), 0.82 (s, 3H, CH₃), 0.80 (s, 3H, CH₃).

### 5. 1-(3β, 5α)-3-Hydroxyandrostan-17-one)-4-(8-hydroxy-5-nitroquinoline)succinate (990624)

The reaction mixture of (3β,5α)-3-Hydroxyaudrostan-17-one monosuccinate (25 mg, 0.064 mmol), 8-hydroxy-5-nitroquinoline (13 mg, 0.070 mmol), EDCI (20 mg, 0.11 mmol), DMAP (2 mg, 0.02 mmol) and dichloromethane (4 ml) was stirred at room temperature for 6 h. Then 20 ml of dichloromethane was added. Organic layer was washed with H₂O, 5% Na₂CO₃, H₂O and brine, and then dried over MgSO₄. After the solvent was removed under vacuum, the residue was separated by column chromatography to give 24 mg 1-((3β,5α)-3-hydroxyandrostan-17-one)-4-(8-hydroxy-5-nitroquinoline)succinate as yellow solid.

The chemical structure analysis was performed by ¹HNMR (CDCl₃, 600 MHz): δ 9.09(d, 1H, Ar—H) 9.01 (d, 1H, Ar—H), 8.45(d, 1H, Ar—H), 7.68(q, 1H, Ar—H), 7.57 (d, 1H, Ar—H), 4.76 (m, 1H, CHO), 3.16 (t, 2H, CH₂), 2.81 (t, 2H, CH₂), 2.43 (m, 1H, H₁₆), 2.20-0.72 (m, 21H,), 1.83 (d, 3H, CH3), 0,94 (s, 3H, CH₃), 0.88 (s, 3H, CH₃).

### 6. 1-((3β,5α)-3-Hydroxyandrostan-17-one)-4-[(−)-scopolamine]succinate (991228)

The reaction mixture of (3β,5α)-3-Hydroxyandrostan-17-one monosuecinate (39 mg, 0.10 mmol), (−)-scopolamine (30 mg, 0.10 mmol), EDCI (30 mg, 0.15 mmol), DMAP (3 mg, 0.028 mmol) and dichloromethane (4 ml) was stirred at room temperature for 20 h. Then 20 ml of dichloromethane was added. Organic layer was washed with H₂O, 5% Na₂CO₃, H₂O and brine, and then dried over MgSO₄. After the solvent was removed under vacuum, the residue was separated by column chromatography to give mg 1-((3β,5α)-3-hydroxyandrostan-17-one)-4-[(−)-scopolamine]succinate, yield.

The chemical structure analysis was performed by ¹HNMR (CDCl₃, 600 MHz): δ 6.81 (s, 1H, Ar—H), 6.54 (s, 1H, Ar—H), 6.39(s, 2H, Ar—H), 5.98(d, 2H, Ar—H), 5.92 (d, 1H, Ar—H), 4.72 (m, 1H, OCH), 4.61 (d, 1H), 4.40 (t, 1H,), 4.19 (t, 1H,) 3.82(s, 3H, OCH3), 3.76 (s, 6H, OCH3), 3.00-0.60 (m, H).

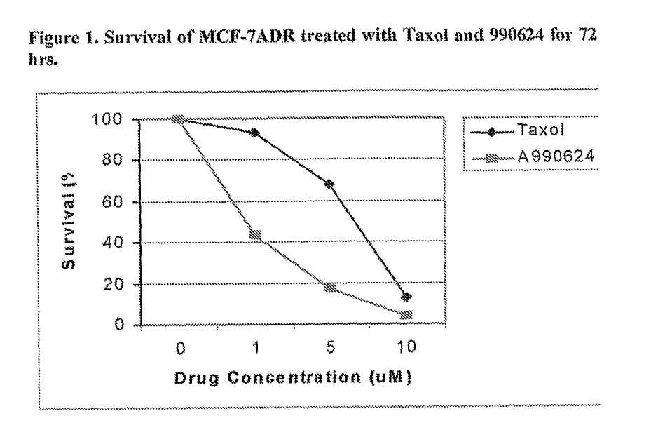

To test the effect of androsterone derivatives of the disclosure, HCT116 cells (colorectal carcinoma ceils) were contacted with various concentrations of the androsterone derivatives of the disclosure. The results are presented in Table 1. In addition, the androsterone derivatives of the disclosure showed increased killing of breast cancer cells having resistance to doxorubicin (MCF-7ADR cells) (see, e.g., FIGURE 1). The derivatives were less toxic to normal cells and provided to be effective at killing cancer cells (particularly breast cancer cells).

TABLE 1
In vitro antitumor activity of androsterone
analogues against HCT116 cells.

| Androsterone | | | | |
|---|---|---|---|---|
| Derivative | 10 μM | 1 μM | 100 nM | 10 nM |
| 990624 | 0 | 100 | | |
| 991022 | 0 | 100 | | |
| 991228 | 0 | 98.29 | | |
| 991120 | 0 | 100 | | |
| 991123 | 0 | 96.97 | | |
| 991027 | 0 | 0 | 4.00 | |
| 5-FU | 17.00 | | | |

### 7. The Compounds of this Example are Prepared Utilizing a camptothecin-based compound

### 7A. Camptothecin-20-O-ester of 4-carboxyphenoxyacetic acid (intermediate compound)

The mixture of camptothecin (100 mg, 0.287 mmol), 4-carboxyphenoxyacetic acid (1.12 mg, 0.57 mmol), EDCI (82 mg, 0,43 mmol), DMAP (10 mg, 0.1 mmol), N,N-dimethylformamide (4 ml) and dichloromethane (4 ml) were stirred in the room temperature for 48 h, then dichloromethane (50 ml) was added to the solution. Organic layer was washed with water (20 ml), saturated NaHCO₃ aqueous solution (20 ml) and brine (20 ml), and then dried over MgSO₄. After the solvent was removed under reduced pressure, the resulting solid was separated by column chromatography (eluent: CHCl₃: C₂H₅OH 5:1) to afford 80 mg camptothecin-20-O-4-fluorophenoxyacetate, yield: 67.0%, mp °(dec).

The chemical structure analysis was performed by ¹HNMR (CDCl₃, 600 MHz): δ 8.40 (s, 1H, Ar—H), 8.30 (d, 1H, Ar—H), 8.07 (m, 2H, Ar—H), 7.95 (d, 1H, Ar—H), 7.86 (t, 1H, Ar—H), 7.67 (t, 1H, Ar—H), 7.25 (s, 1H, Ar—H), 6.96 (m, 2H, Ar—H), 5.68 (d, 1H, H17), 5.43 (d, 1H, H17), 5.29 (s, 2H, H5), 4.91 (q, 2H, OCH₂CO), 2.25 (dm, 2H, CH₂), 0.97 (t, 3H, CH₃).

### 7B. Camptothecin-20S-O-(4-carboxyphenoxyacetate) linked with epiandrosterone (0103021)

The mixture of camptothecin-20S-O-(4-carboxyphenoxyacetate) (10 mg, 0.019 mmol), epiandrosterone (11 mg, 0.038 mmol), EDCI (25 mg, 0.13 mmol), DMAP (2 mg, 0.02 mmol), and dichloromethane (3 ml) were stirred in the room temperature for 24 h, then dichloromethane (20 ml) was added to the solution.

Organic layer was washed with water (20 ml), saturated NaHCO₃ aqueous solution (10 ml) aid brine (20 ml), aid then dried over MgSO₄. After the solvent was removed under reduced pressure, the resulting solid, was separated by column chromatography (eluent: ethyl acetate-ethanol 9:1) to afford 2.0 mg target compound.

The chemical structure analysts was performed by ¹HNMR (CDCl₃, 600 MHz): δ 8.41 (s, 1H, Ar—H), 8.27 (d, 1H, Ar—H), 7.98 (m, 2H, Ar—H), 7.86 (t, 1H, Ar—H), 7,69 (t, 1H, Ar—H), 7.29 (s, 1H, Ar—H), 7.24 (s, 1H, Ar—H), 7.21 (s, 1H, Ar—H), 6.95 (d, 1H, Ar—H), 5.68 (d, 1H, CPT-H17), 5.42 (d, 1H, CPT-H17), 5.29 (s, 2H, CPT-H5), 4.89 (q, 2H, OCH₂CO), 4.23 (m, 1H, epiandrosierone-H3), 2.60-1.00 (m, 30H, CPT-H18 and epiandrosterone-H).

### 7C. Camptothecin-20S-O-(4-carboxyphenexyacetate) linked with androsterone (010216)

The mixture of camptothecin-20S-O-(4-carboxyphenoxyacetate) (10 mg, 0.019 mmol), androsterone 11 mg, 0.038 mmol), EDCI (25 mg, 0.13 mmol), DMAP (2 mg, 0.02 mmol), and dichloromethane (3 ml) were stirred in the room temperature for 24 h, then dichloromethane (20 ml) was added to the solution. Organic layer was washed with water (20 ml), saturated NaHCO₃ aqueous solution (10 ml) and brine (20 ml), and then dried over MgSO₄. After tire solvent was removed under reduced pressure, the resulting solid was separated by column chromatography (eluent: ethyl acetate-ethanol 9:1) to afford 4.3 mg solid.

The chemical structure analysis was performed by ¹HNMR (CDCl₃, 600 MHz): δ 8.41 (s, 1H, Ar—H), 8.28 (d, 1H, Ar—H), 7.99 (m, 3H, Ar—H),7.87 (t, 1H, Ar—H), 7.69 (t, 1H, Ar—H), 7.35 (s, 1H, Ar—H), 7.21 (s, 1H, Ar—H), 6.96 (d, 2H, Ar—H), 5.71 (d, 1H, CPT-H17), 5.43 (d, 1H, CPT-H17), 5.29 (s, 2H, CPT-H5), 4.90 (q, 2H, OCH₂CO), 4.13 (m, 1H, androsterone-H3), 3.00-2.00(m, 21H, CPT-H18 and androsterone-H), 1.28 (s, 6H, CH₃), 0.97 (t, 3H, CPT-H19).

Many camptothecin-based compounds are generally available in the art and would be known to one of ordinary skill in the art. Some examples of such camptothecin-based compounds include: (20S)-9-nitro CPT; (20S)-7-chloro-n-propyldimethyisilyl CPT; (20S)-10-hydroxy-7-chloro-n-propyldimethylsilyl CPT; (20S)-10-acetoxy-7-chloro-n-propyldimethylsily CPT; (20S)-7-tert-butyldimethylsily CPT; (20S)-10-hydroxy-7-tert-butyldimethylsilyl CPT; (20S)-10-acetoxy-7-tert-butyldimethylsilyl CPT; (20S)-9-hydroxy CPT; (20S)-9-amino CPT; (20S)-10-amino CPT; (20S)-9-amino-10-hydroxy CPT; (20S)-9-methylamino CPT; (20S)-9-chloro CPT; (20S)-9-fluoro CPT: (20S)-9-piperidino CPT; (20S)-9-morpholinomethyl CPT; (20S)-9,10-dichloro CPT; (20S)-10-bromo CPT; (20S)-10-chloro CPT; (20S)-10-methyl CPT; (20S)-10-fluoro CPT; (20S)-10-nitro CPT; (20S)-10,11-methylenedioxy CPT; (20S)-10-formyl CPT; (20S)-10-nonylcarbonyloxy CPT; (20S)-10-undecylcarbonyloxy CPT; (20S)-10-heptadecylcarbonyloxy CPT; (20S)-10-nonadecylcarbonyloxy CPT; (20S)-9-nitro-10,11-methylenedioxy CPT; (20S)-9-(4-methylpiperazinylmethyl)-10-hdroxy (CPT); 20S)-9-[4-(1-piperidino)-1-piperidinomethyl]-10-hydroxy CPT; (20S)-9-methyl-10,11-methylenedioxy CPT; (20S)-9-chloro-10,11-methylenedioxy CPT; (20S)-9-cyano-10,11-methylenedioxy CPT; (20S)-9-acetoxy-10,11-methylenedioxy CPT; (20S)-9-acetylamino-10,11-methylenedioxy CPT; (20S)-9-aminomethyl-10-hydroxy CPT; (20S)-9-ethoxymethyl-10-hydroxy CPT; (20S)-9-methylaminomethyl-10-hydroxy CPT; (20S)-9-n-propylaminomethyl-10-hydroxy CPT; (20S)-9-dimethylaminomethyl-10-hydroxy CPT; (20S)-9-cyclohexylaminomethyl-10-hydroxy CPT; (20S)-9-(2-hydroxyethyl)aminomethyl-10-hydroxy CPT; (20S)-9-(trimethylamino)methyl-10-hydroxy CPT, methanesulfonate; (20S)-9)-morpholinomethyl-10-hydroxy CPT; (20S)-9-cyanomethyl-10-hydroxy CPT; (20S)-CPT-7-aldehyde; (20S)-10-methoxy CPT-7-aldehyde; (20S)-7-acetoxymethyl CPT; (20S)-7-acetoxymethyl-10-methyl CPT; (20S)-7-cyano-10-methoxy CPT; (20S)-7-cyano CPT; (20S)-7-formylethenyl CPT; (20S)-7-ethoxycarbonylethenyl CPT; (20S)-7-cyanoethenyl CPT; (20S)-7-(2,2-dicyanoethenyl) CPT; (20S)-7-(2-cyano-2-ethoxycarbonyl)ethenyl CPT; (20S)-7-ethoxycarbonylethyl CPT; (20S)-7-ethyl CPT; (20S)-7-n-propyl CPT; (20S)-7-acetoxymethyl CPT; (20S)-7-n-propylcarbonyloxymethyl CPT; (20S)-7-ethoxycarbonyl CPT; (20S)-7-ethyl-10-hydroxy CPT; (20S)-7-ethyl-10-acetyloxy CPT; (20S)-7-methyl-10-aminocarbonyloxy CPT; (20S)-7-n-propyl-10-piperidinocazbonyloxy CPT; (20S)-7-ethyl-10-(2-dimethylamino)ethyl CPT; and (20S)-7-ethyl-10-carbamoyloxy derivatives of CPT such as (20S)-7-ethyl-10-[4(1-piperidino)-piperidino carbonyloxy CPT; (20S)-7-ethyl-10-(1-piperazine)carbonyloxy CPT; (20S)-7-ethyl-10-(4-i-propylaminocarbonylmethylpiperazine)carbonyloxy CPT; (20S)-7-ethyl-10-[4(1-pyrrolidinyl)piperazine]carbonyloxy CPT; (20S)-7-ethyl-10-[(4-dimethylamino)-1-piperidino]carbonyloxy CPT: (20S)-7-ethyl-10-[4-(di-n-propylamino)-1-piperidinol]carbonyloxy CPT; (20S)-7-ethyl-10-[(4-(di-n-butylamino)-1-piperidino]carbonyloxy CPT; (20S)-7-ethyl-10-[4-(1-pyrrolidino)-1-piperidino)]carbonyloxy CPT; (20S)-7-ethyl-10-[(4-(1-piperidino)-1-piperidino]carbonyloxy CPT; (20S)-7-ethyl-10-[N-methyl-N-2-(dimethylamino)ethylamino]carbonyloxy CPT; (20S)-7-(tert-butyldimethylsilyl)CPT; (20S)-7-(tert-butoxyiminomethyl) CPT (Gimatecan); (20S)-7-butyl-10,11-methylenedioxy CPT; (20S)-7-bromomethyl-10-hydroxy OPT; (20S)-7-butyl-10-amino CPT; (20S)-7-(tert-butyldimethylsilyl)-10-hydroxy CPT; (20S)-7-[2-trimethylsilyl)ethyl)]CPT (Karentican); (20S)-7-[(4-fluorophenoxy)acetyloxymethyl]CPT; (20S)-7-[(4-methoxyphenoxy)acetyloxymethyl]CPT; (20S)-7-((4-cyano-3-fluorophenoxy)acetyloxymethyl]CPT; (20S)-7-[(3,4,5-trimethoxyphenyl)acetyloxymethyl]CPT; (20S)-10-[(4-cyano-3-fluorophenoxy)acetyloxy]CPT; (20S)-10-[(3,4,5-trimethoxyphenyl)acetyloxy]CPT; (20S)-7-(4-methylpiperazinomethylene)-10,11-ethylenedioxy CPT (Exatecan); (20S)-7-[2-(N-isopropylamino)ethyl]CPT (Belotecan): (20S)-[5(RS)-2-hydroxyethoxy)]CPT (21); and combinations thereof.

The compounds of this example are prepared by reacting any androsterone analog or any epiandrosterone analog.

One of skill, in the art will, recognize that other similar androsterone analog or epiandrosterone analog may be obtained from commercial sources or prepared by art-recognized procedures to he used in these steps to prepare compounds of this disclosure. By reacting a compound shown in the list of camptomecin-based analogs with an androsterone analog in accordance with the guidelines for reaction condition, compounds of the disclosure will be obtained. These compounds will exhibit the desired characteristics to a greater or lesser extent

To test the effect of androsterone derivatives of this example, HCT116cells (colorectal carcinoma cells) were plated in 60 mm Petri dishes containing 2.7 ml of medium (modified McCoy's 5a medium containing 10% fetal bovine serum and 100 units/ml penicillin and 100 □g/ml streptomycin). The cells were incubated in a CO₂ incubator at 37° C. for 5 hours for attachment to the bottom of Petri dishes. Drugs were made up fresh in medium at ten times the final concentration, and then 0.3 ml of tins stock solution was added to the 2.7 ml of medium containing 5% bovine calf serum (BCS) in the dish. The cells were then incubated with drugs for 72 hours at 37° C. At the end of incubation the drug-containing media were decanted, the dishes were rinsed with 4 ml of Hank's Balance Salt Solution (HBSS). 5 ml of fresh medium containing 15% BCS was added, and the dishes were returned to the incubator for colony formation. The cell colonies stained with methylene blue (0.5% in ethanol) were counted using colony counter after incubation for 8 days for HCT116 cells. Cell survival was calculated and the values of IC50 (the drug concentration producing 50% inhibition of colony formation) were determined for each tested compound. Tire values of IC50 were 2.5 nM for 010216 and 3.5 nM for 0100321. The results are presented in Table 2.

TABLE 2
Cell survival of HCT116 cells treated with 010216 and 0103021

| Drug | % Survival | % Survival |
|---|---|---|
| conc. (nM) | For 010216 | For 0103021 |
| 0 | 100 | 100 |
| 1 | 91 | 100 |
| 5 | 2 | 24 |
| 10 | 0 | 0 |

A number of embodiments have been described. Nevertheless, it will be understood that various modifications may be made without departing from, the spirit and scope of the description. Accordingly, other embodiments are within the scope of the following claims.

1. A compound comprising the general formula II: 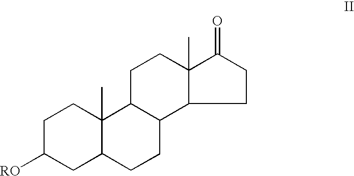
wherein R is selected from the group consisting of an aromatic, an aromatic nitrogen-containing heterocycle, and a non-aromatic nitrogen-containing heterocycle.

2. The compound of claim 1, wherein the R group has an aromatic system with electron withdrawing groups.

3. The compound of claim 1, wherein the aromatic nitrogen-containing heterocycle is a 5- or 6-membered monocyclic substituent or a bicyclic fused or linked 5- or 6-membered ring.

4. The compound of claim 3, wherein the aromatic nitrogen-containing heterocycle is an imidazolyl, an indolyl, a pyridinyl, a pyrimidinyl, a pyrrolyl, a quinolinyl, a tetrazolyl, and a 1,2,4-triazolyl.

5. The compound of claim 3, wherein the aromatic nitrogen-containing heterocycle is selected from the group consisting of 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, and 2,4,6-trimethylpyridine.

6. The compound of claim 1, wherein the non-aromatic nitrogen-containing heterocyle comprises a 4- to 6-membered rings selected from the group consisting of an acetimido, a morpholinyl, a lactam, an imide, a phthalimido, a piperidyl, a piperidino, a piperazinyl, a pyrrolidinyl, and a succinimido.

7. The compound of claim 6, wherein the imide is selected from the group consisting of γ-butyrolactam, ε-caprolactam, N-phenyl-β-propiolactam.

8. The compound of claim 1, wherein the non-aromatic nitrogen-containing heterocycle is selected from the group consisting of a 1,2-dimethylpiperidine, a 2,5-dimethylpiperazine, a 1,2-dimethylpyrrolidine, a 1-ethylpiperidine, a n-methylpyrrolidine, a morpholine, a piperazine, a piperidine, a pyrrolidine, a 2,2,6,6-tetramethylpiperidine, and a 2,2,4-trimethylpiperidine.

9. The compound of claim 1, wherein the R group is an atropine or a scopolamine.

10. The compound of claim 1, wherein the R group is a camptothecin analog.

11. The compound of claim 1, wherein the R group is selected from the group consisting of: 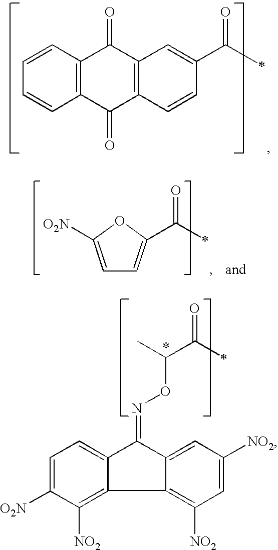
wherein * indicates the (+) chiral center in the original molecule.

12. A compound comprising the general formula III: 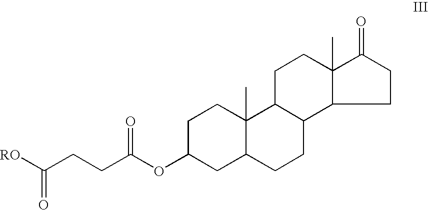
wherein R is selected from the group consisting of an aromatic, an aromatic nitrogen-containing heterocycle, and a non-aromatic nitrogen-containing heterocycle.

13. The compound of claim 12, wherein the R group has an aromatic system with electron withdrawing groups.

14. The compound of claim 12, wherein the aromatic nitrogen-containing heterocycle is a 5- or 6-membered monocyclic substituent or a bicyclic fused or linked 5- or 6-membered ring.

15. The compound of claim 14, wherein the aromatic nitrogen-containing heterocycle is an imidazolyl, an indolyl, a pyridinyl, a pyrimidinyl, a pyrrolyl, a quinolinyl, a tetrazolyl, and a 1,2,4-triazolyl.

16. The compound of claim 14, wherein the aromatic nitrogen-containing heterocycle is selected from the group consisting 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, and 2,4,6-trimethylpyridine.

17. The compound of claim 12, wherein the non-aromatic nitrogen-containing heterocyle comprises a 4- to 6-membered rings selected from the group consisting of an acetimido, a morpholinyl, a lactam, an imide, a phthalimido, a piperidyl, a piperidino, a piperazinyl, a pyrrolidinyl, and a succinimido.

18. The compound of claim 17, wherein the imide is selected from the group consisting of γ-butyrolactam, ε-caprolactam, N-phenyl-β-propiolactam.

19. The compound of claim 12, wherein the non-aromatic nitrogen-containing heterocycle is selected from the group consisting of a 1,2-dimethylpiperidine, a 2,5-dimethylpiperazine, a 1,2-dimethylpyrrolidine, a 1-ethylpiperidine, a n-methylpyrrolidine, a morpholine, a piperazine, a piperidine, a pyrrolidine, a 2,2,6,6-tetramethylpiperidine, and a 2,2,4-trimethylpiperidine.

20. The compound of claim 12, wherein the R group is an atropine or a scopolamine.

21. The compound of claim 12, wherein the R group is a camptothecin analog.

22. The compound of claim 12, wherein R is selected from the group consisting of:

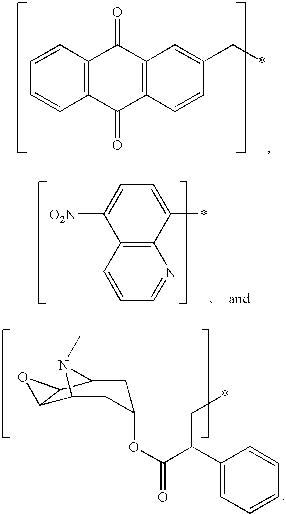

23. A pharmaceutical composition comprising the compound of claim 1 or 12 in a pharmaceutically acceptable carrier.

24. A method of treating an androgen-associated disease or disorder or an estrogen-associated disease or disorder comprising administering to a subject in need of such treatment a therapeutically effective amount of a compound of claim 1 or 12.

25. The method of claim 24, further comprising administering to the subject a therapeutically effective amount of an agent selected from the group consisting of an antiestrogen or an antiandrogen.

26. A method of treating or reducing the risk of developing breast cancer comprising administering to a subject in need of such treatment a therapeutically effective amount of a compound of claim 1 or 12 or a pharmaceutical composition thereof.

27. The method of claim 26, further comprising administering to the subject a therapeutically effective amount of an antiestrogen agent.

28. The method of claim 26, further comprising administering a therapeutically effective amount of an LHRH agonist or antagonist.

29. The method of claim 26, further comprising administering a therapeutically effective amount of a second androgenic compound.
